(19) United States
(12) Patent Application Publication
(10) Pub. No.: US 2017/0347960 A1
(43) Pub. Date: Dec. 7, 2017

(54) MOBILE NEWBORN CARE BED AND METHODS OF NEWBORN CARE

(71) Applicant: General Electric Company, Schenectady, NY (US)

(72) Inventors: Steven Mitchell Falk, Baltimore, MD (US); Karen P. Starr, Monkton, MD (US); Matthew L. Riesser, Brighton, MI (US)

(73) Assignee: General Electric Company, Schenectady, NY (US)

(21) Appl. No.: 15/174,106

(22) Filed: Jun. 6, 2016

(51) Int. Cl. A61B 5/00; A61B 5/0205; A61B 5/08; A61B 5/11; A61G 11/00; A61M 16/08; A61M 16/06; A61F 7/00; A61B 5/083; A61M 16/00; A61B 5/087; A61B 5/0245; A61B 5/1455

(52) U.S. Cl. CPC A61B 5/6892; A61M 16/08; A61B 5/0205; A61B 5/0826; A61B 5/1102; A61B 5/7282; A61B 5/7425; A61G 11/00; A61M 16/06; A61B 5/0022; A61B 5/0833; A61B 5/0836; A61B 2503/045; A61B 2562/0247; A61M 2016/003; A61B 5/087; A61B 5/0245; A61B 5/14552; A61F 2007/0088

(57) ABSTRACT

A mobile newborn care bed is configured to be positioned at a delivery location of an infant includes a bassinet containing a mattress for receiving the infant and a frame that supports the bassinet. At least two capacitive sensors are incorporated in the mattress that record cardiac signals, and an on-bed computing system is configured to receive the cardiac signals and determine a heart rate for the infant. A battery supported by the frame powers the on-bed computing system, and a digital display is communicatively connected to the on-bed computing system and displays the heart rate.

### BACKGROUND

The present disclosure relates to the field of infant care, and more specifically to systems and methods for providing diagnosis and therapy to newborn infants.

At the time of birth, infants need immediate assessment and care, including assessment of heart and respiratory function. Infant patients can experience relatively rapid changes in condition, especially immediately after birth. Depending on the infant's condition, various therapies may be provided, including resuscitation or other respiratory care. Further, infants also often require a heated environment to prevent cold stress. Accordingly, the use of radiant warmers and/or incubators for maintaining environmental conditions suitable for an infant is common immediately after an infant's birth. Conventional warming devices comprise a bassinet provided underneath a warming element, which is suspended directly over the infant.

According to long-standing care standards, an infant's umbilical cord was cut immediately upon delivery and the infant was removed from the mother to check vital signs and provide any needed therapy, such as respiratory therapy. In such instances, babies were removed from the delivery location and placed on a bassinet or infant bed, often containing a radiant warmer. Currently available infant beds and radiant warmers are configured to be positioned in a corner of a delivery room so as not to crowd the space next to the mother. Moreover, most infant care beds and radiant warmers are one, integrated, bulky device, where the bassinette is built into the warmer. Resuscitation equipment and/or monitoring equipment, if any, is positioned near the infant bed/radiant warmer away from the delivery location.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment a mobile newborn care bed is configured to be positioned at a delivery location of an infant includes a bassinet containing a mattress for receiving the infant and a frame that supports the bassinet. At least two capacitive sensors are incorporated in the mattress that record cardiac signals, and an on-bed computing system is configured to receive the cardiac signals and determine a heart rate for the infant. A battery supported by the frame powers the on-bed computing system, and a digital display is communicatively connected to the on-bed computing system and displays the heart rate.

One embodiment of a method of monitoring a newborn infant includes recording cardiac signals from the infant with two or more capacitive sensors incorporated in the mattress. The recorded cardiac signals are received at an on-bed computing system of the mobile newborn care bed, and a heart rate is determined in the on-bed computing system based on the cardiac signals. The heart rate is then displayed on a digital display communicatively connected to the on-bed computing system.

Another embodiment of a mobile newborn care bed is configured to be positioned at a delivery location of a newborn infant includes a bassinet containing a mattress that supports the infant and a frame that supports the bassinet. The mobile newborn care bed further includes an on-bed computing system comprising a processor, a flow sensor communicatively connected to the on-bed computing system, and a CO₂ sensor communicatively connected to the on-bed computing system. A resuscitation module is executable on the processor to receive a flow measurement measured by the flow sensor and a CO₂ measurement measured by the CO₂ sensor and determine one or more respiration parameters for the infant. A digital display is communicatively connected to the on-bed computing system and displays one or more of the respiration parameters.

A method of providing resuscitative care to a newborn infant includes measuring a gas flow with a flow sensor in a breathing circuit for the infant. The flow measurement is communicated to an on-bed computing system. An expired CO₂ is measured with a CO₂ sensor in the breathing circuit for the infant, and the CO₂ measurement is communicated to the on-bed computing system. One or more respiration parameters are determined for the infant with the on-bed computing system based on at least the flow measurement and the CO₂ measurement. One or more of the respiration parameters may then be displayed on a digital display communicatively connected to the on-bed computing system.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a schematic diagram of one embodiment of a mobile newborn care bed and separate, freestanding warmer configured to pair with the mobile newborn care bed.

FIG. 2 is a schematic system diagram of an on-bed computing system and sensor elements for a mobile newborn care bed, as well as a hub device and host network communicatively connected to the on-bed computing system.

FIG. 3 is a schematic diagram depicting one embodiment of an on-bed computing system.

FIG. 4 is a schematic diagram depicting one embodiment of a hub computing system.

FIG. 5 depicts one embodiment of a method of monitoring a newborn infant.

FIG. 6 depicts one embodiment of a method of providing resuscitative care to a newborn infant.

### DETAILED DESCRIPTION

Newborn birth and delivery care standards are trending towards maintaining the infant at the birthing site to the extent possible in order to allow delayed cord clamping and cutting for several minutes so that the blood in the umbilical cord is transferred to the baby. Accordingly, through their experimentation and research in the relevant field, the present inventors have recognized that such delated cord clamping and other modern care standards for newborn infants immediately after birth have made current radiant warmer and resuscitation platform technology obsolete. The inventors have recognized that a device is needed to provide diagnosis and therapy to a newborn infant next to the mother and at the site of birth so that such therapy can be administered before and/or during the cord clamping. Further, the inventors have recognized that devices and systems are needed that provide monitoring and resuscitation care for infants easily and with minimal attachment of devices to the baby. Further, the inventors have recognized that devices and systems are needed that provide immediate and accessible display of multiple relevant vital parameters, such as cardiac and respiration parameters, to clinicians providing care, and also seamless transmission and storage and of such data to the patient's healthcare records.

In light of their experimentation and research in the relevant field, the present inventors have further recognized that clinicians providing care to infants at birth are often seeking more guidance for providing safe respiratory and resuscitative care to infants, such as to reduce barotrauma and volutrauma, and to reduce or delay use of invasive ventilation as much as possible in the delivery room. In light of these problems and needs in the relevant field recognized by the inventors, they developed the disclosed mobile newborn care bed including built-in, non-contact cardiac monitoring and non-invasive respiratory therapy devices for providing positive pressure ventilation and/or continuous positive airway pressure.

In further view of their recognition of problems and needs in the relevant field, the inventors developed the mobile newborn care bed and associated methods of infant monitoring and care. The mobile newborn care bed is configured to be positioned at a delivery location of an infant and is equipped with built-in diagnosis and therapy equipment and systems to enable a clinician to provide monitoring and/or resuscitative care to an infant immediately upon birth, including before and during cord clamp.

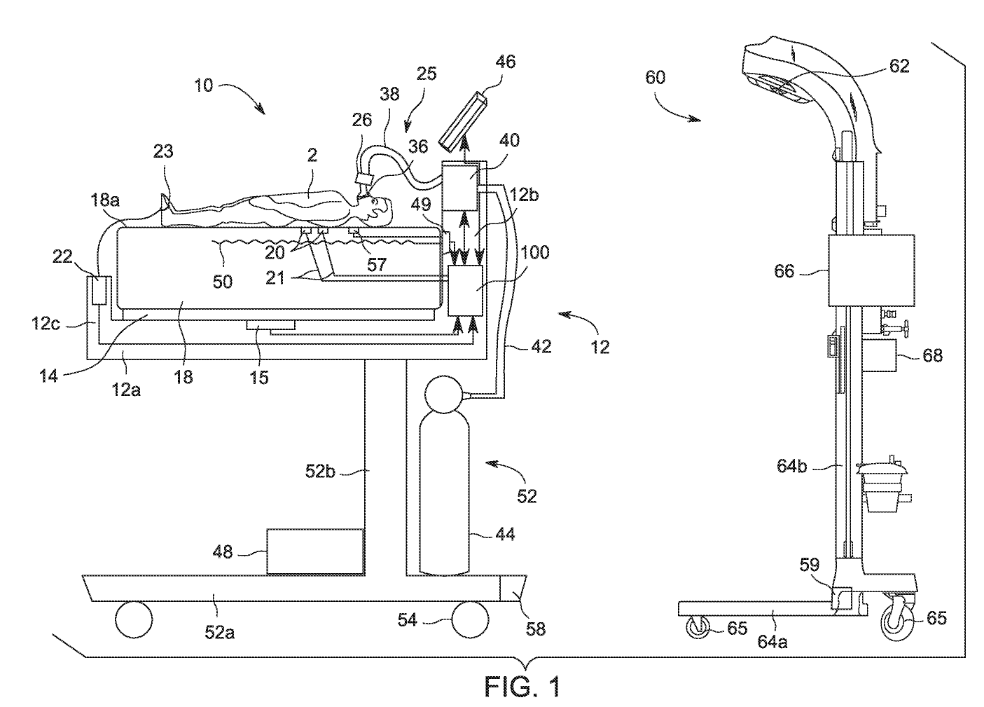

FIG. 1 depicts one embodiment of a mobile newborn care bed 10, which is relatively small and agile and able to be positioned at a delivery location of an infant. The mobile newborn care bed has a bassinet 12 containing a mattress 18 on which the infant 2 is placed. The mattress 18 is preferably a flat or slightly concave cushioned surface, but can be any flat or curved surface capable of receiving the infant 2. A frame 52 is underneath the bassinet 12 and supports the bassinet 12. The frame includes a base frame portion 52a connecting to one or more wheels 54 that allow the mobile newborn care bed 10 to be easily moved. The frame 52 also includes a vertical frame portion 52b that elevates and attaches to the bassinet 12. In various embodiments, the vertical frame portion 52b may be adjustable to adjust the height of the bassinet 12. The base frame portion 52a may be configured to support various elements comprising part of the mobile newborn care bed 10, such as one or more batteries 48 and/or gas supply tanks 44.

In the depicted embodiment, the bassinet 12 includes a bottom portion 12a supporting the mattress 18, and also includes a head portion 12b adjacent to one side of the mattress 18 and a foot portion 12c a pulse oximeter device 22. In other embodiments, such devices may be housed or incorporated at other locations on the mobile newborn care bed 10 or may be provided separately but in conjunction with the mobile newborn care bed 10. In still other embodiments, such devices may be separately housed and attachable to or otherwise associated with the bassinet 12 and/or frame 52.

In certain embodiments, the bassinet 12 may include a rigid base plate 14 immediately underneath and supporting the weight of the mattress 18. At least one load cell 15 is positioned under the base plate in order to sense a load of the infant and provide a load measurement 84 from which a weight can be calculated. Specifically, the load cell(s) 15 and associated electronic weighing system may be calibrated to the weight of the mattress 18 so that the weight of an infant 2 can be determined immediately upon placing the infant 2 on the mattress 18. Furthermore, other parameters may also be determined based on continuous load measurement by the one or more load cells 15, including whether the infant is breathing or is experiencing an apnea event. In one embodiment discussed in more detail hereinbelow, the load measurements 84 may be received and processed by a load module 74, which is a software module stored on and executed by the on-bed computing system 100 to determine weight 85, and detect and apnea event and generate an apnea event notification 86.

The mobile newborn care bed 10 may further include sensors incorporated into the mattress 18 to measure cardiac signals from the infant 2 when the infant is placed on the mattress 18. Specifically, two or more capacitive sensors 20 may be incorporated into a top portion of the mattress 18. For example, the capacitive sensors 20 may be metal plates positioned on a top surface 18a of the mattress 18, or just beneath a mattress cover, such as between a cushion filling of the infant mattress 18 and a cover for the cushion. In various other embodiments, the capacitive sensors 20 may be embedded close to the top surface 18a of the mattress, such as imbedded in a foam or other cushion material of the mattress. In still other embodiments, two or more capacitive sensors 20 may be integrated into or fixed to a cover or casing of the mattress 18. The two or more capacitive sensors 20 record the time varying bioelectric field produced by the infant's beating heart, which imparts corresponding changes on the metal plate by capacitive transmission. Accordingly, the capacitive sensors 20 detect and measure the electric field without any direct physical contact between the sensors and the infant's skin, and no electrically conducting path between the sensors 20 and the infant is needed.

Thereby, cardiac signals 81 may be recorded from the infant 2 immediately upon placing the infant 2 on the mattress 18, and can be continually recorded while the infant 2 remains on the mattress without any need for attaching physical ECG electrodes to the infant's skin. In one exemplary embodiment, the mattress 18 may incorporate a grid of capacitive sensors 20, such as a grid of 4, 6, 9, 12, or more capacitive sensors 20, various ones of which may provide the best measurement of the bioelectric fields and thus cardiac signals 81 for the infant, depending on the position of the infant 2 on the mattress 18. The capacitive sensors 20 may be connected to the on-bed computing system 100 by leadwires 21, which may be embedded in the cushion of the mattress 18 or in a casing thereof. The leadwires 21 may have a connector that connects to a mating connector on the bassinet 12 of the mobile newborn care bed 10, or by wireless means via a wireless transmitter in the mattress 18 and corresponding wireless receiver incorporated into the bassinet 12.

A heart rate and other cardiac information 82 may be determined from the capacitively measured cardiac signals 81. The capacitive sensors 20 may connect to a signal processing module including hardware and software to receive the measured signals from the capacitive sensors 20 and create cardiac signals 81 that may be used by the system for medical assessment and diagnosis purposes. Such signal processing circuitry and software may include an analog to digital converter, filters, amplifiers, and the like. Such circuitry and software may be provided in a stand alone module contained on the mobile newborn care bed 10 and connecting to the on-bed computing system 100, or may be incorporated into the on-bed computing system 100 as described further herein.

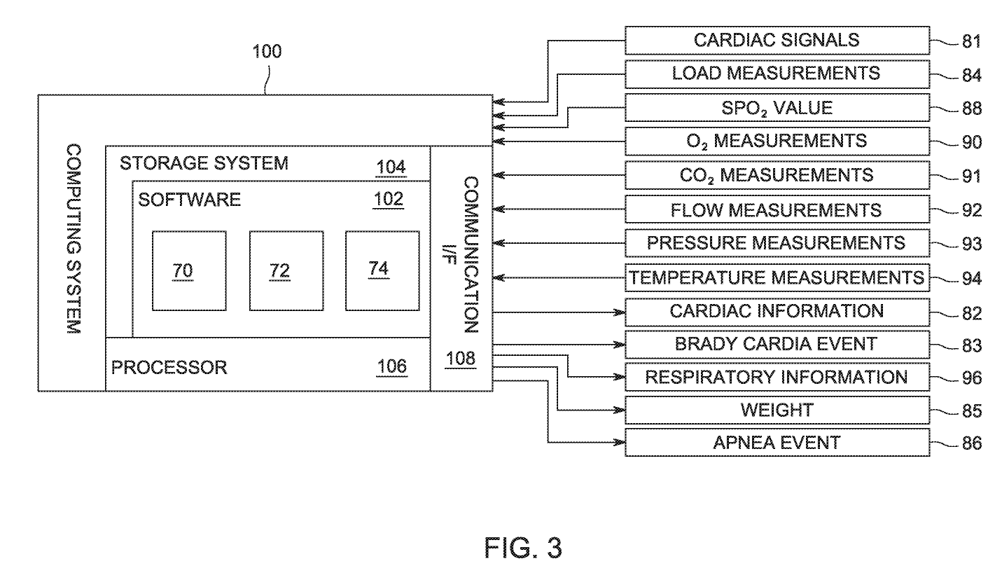

In the depicted embodiment, the on-bed computing system 100 receives the cardiac signals 81 (FIG. 3) and processes the cardiac potential measurements to determine cardiac information 82. For example, the on-bed computing system 100 may have a software module for processing the digitized cardiac signals 81, a cardiac module 70, to determine various cardiac information 82 about the cardiac health of the infant 2, such as heart rate and/or wave morphology information. In one embodiment, the cardiac module 70 includes instructions executable to detect R-waves within the cardiac signals 81, from which the heart rate can be determined.

The cardiac module 70 may further be configured to detect a bradycardia event based on the cardiac potential measurements, which will be recognized by a person having ordinary skill in the art as important information for determining the health and well-being of an infant 2 within its first few hours and days after birth. For instance, as explained in more detail hereinbelow, the cardiac module 70 may continually determine heart rate and detect a bradycardia event when the heart rate is below a predetermined threshold value, and then generate a bradycardia event notification 83. The cardiac information 82 and/or bradycardia event notification 83 may then be transmitted from the on-bed computing system 100 to a hub device 68 and/or to a host network 76, such as to a network of a hospital, for storage in a patient medical record database 78. The cardiac module 70 may further store the cardiac information 82 and/or bradycardia event notifications 83 in storage system 104 of the on-bed computing system 100, and may also provide further cardiac assessment functions, such as heart rate trending assessment or other time varying data to plot or otherwise convey variance of the heart rate or other cardiac information 82 over a period of time.

The on-bed computing system 100 may be communicatively connected (i.e. connected by physical or wireless means so as to be able to communicate information to or with another device) to a digital display 46 to communicate display commands thereto, such as to display the cardiac information 82 and/or respiratory information 96 thereon. Accordingly, the digital display 46 associated with the mobile newborn care bed 10 may display the infant's heart rate and/or other cardiac information 82 to a clinician while the clinician is providing medical care to the infant 2. Likewise, the on-bed computing system 100 may control the digital display 46 to display notifications of poor cardiac health or cardiac events, such as to provide a visual alert when a bradycardia event is detected.

The digital display 46 may be any digital display device known in the art and may be fixed to the bassinet 12, such as to the head portion 12b of the bassinet 12, in a way that is visible to clinicians providing care to the infant 2. Alternatively, the digital display 46 may be a separable or completely separate device from the bassinet 12, such as a tablet or mobile computer. In still other embodiments, the digital display 46 may be a display of another device networked with the mobile infant care bed 10, such as the display of the fetal monitor.

As described herein, the digital display 46 may be controlled by the on-bed computing system 100 to provide various health information for the infant, including the respiratory information 96, cardiac information 82, weight 85, arterial oxygen saturation (SpO₂) value 88, temperature measurement 94, or any other relevant value. Additionally, the digital display 46 may provide a user input device, such as via a touchscreen, to provide control input to the on-bed computing system 100 and/or any other system or device on the mobile newborn care bed 10. Accordingly, in various embodiments, multiple systems and devices on or incorporated into the mobile newborn care bed may connect directly to the digital display 46 and be capable of providing control signals to the digital display 46. For example, the ventilator device 40 may connect to the digital display 46 and the digital display 46 may provide a user interface to control the ventilator device 40. Such connectivity may be provided directly between the ventilator device 40 and the digital display 46, or may be routed through the on-bed computing system 100, which may provide a central control for all devices on the mobile newborn care bed, including the ventilator device 40.

The mobile newborn care bed 10 may further include a pulse oximeter sensor device, including an SpO₂ sensor 23 attachable to the infant and connecting to a pulse oximeter 22 that determines an SpO₂ value 88, and transmits the SpO₂ value 88 to the on-bed computing system 100. The pulse oximeter 22 may transmit the SpO₂ value by wired or wireless means, various examples of which are provided herein. The pulse oximeter 22 may be incorporated into the bassinet 12, such as in the foot portion 12c of the bassinet 12 as depicted in FIG. 1. In other embodiments, the pulse oximeter may be a separate device that may be kept in proximity of the bassinet 12 and may be wirelessly paired with the on-bed computing system 100. The SpO₂ sensor 23 may be any sensor device capable of measuring the infant's peripheral oxygen saturation, many varieties of which are well-known in the relevant art, such as a disposable adhesive sensor device configured to wrap around the infant's foot. The SpO₂ sensor 23 may include a wire connecting to the pulse oximeter 22. In still other embodiments, the physical circuitry and software of the pulse oximeter 22 may be incorporated within the on-bed computing system 100, and thus the SpO₂ sensor 23 may communicate measurements related to O₂ saturation directly to the on-bed computing system 100 for determination of SpO₂ values 88 for the infant 2. Upon receipt or determination of the SpO₂ value 88 for the infant 2, the on-bed computing system 100 may transmit the SpO₂ value 88 to the hub device 68, or directly to a host network 76. Further, the on-bed computing system 100 may send control signals to the digital display 46 in order to display the SpO₂ value 88 thereon. Alternatively or additionally, the mobile newborn care bed 10 may incorporate a co-oximeter device that measures and determines one or more of carboxyhemoglobin saturation (SpCO), methemoglobin saturation (SpMet), and/or total hemoglobin concentration (g/dl SpHb). For instance, the co-oximeter device may be a Rainbow SET Pulse CO-Oximeter by Masimo Corporation of Irvine, Calif.

The mobile newborn care bed 10 further includes and incorporates devices and systems for providing resuscitation and other respiratory therapy to an infant 2 in need of such intervention. A person having ordinary skill in the relevant art will know that it is not uncommon for a newborn infant 2 to need some sort of respiratory care or resuscitation immediately after birth, and such care needs to be provided at the location of birth if cord clamping is to be delayed so that the infant 2 can receive the cord blood. Accordingly, the mobile newborn care bed, which as described above, is configured to be positioned at the delivery location of an infant, is equipped with a breathing circuit that includes a ventilator device 40, such as a continuous positive airway pressure (CPAP) device, a positive pressure ventilation (PPV) device, or a positive end-expiratory pressure (PEEP) device (or a ventilator device providing all three respiratory therapies). In the depicted embodiment, the ventilator device 40 receives a gas supply from supply tube 42 connected to gas supply tank 44, which is supported on the base frame portion 52a. The ventilator device 40 regulates the gas supply as appropriate to provide resuscitative or respiratory assistance to the infant 2. The ventilator device 40 connects to the breathing tube 38, which supplies gas to the infant through a mask 36 applied over the infant's nose and mouth, thereby forming breathing circuit 25. In other embodiments, the breathing tube 38 may deliver gas to the infant 2 via a nasal cannula or by some other delivery means.

The breathing circuit 25 is equipped with sensors for measuring parameters relevant to the infant's respiration, which may be provided in the mask 36, breathing tube 38, or at the connection of the mask 36 and the breathing tube 38. Various sensors may be incorporated into the breathing circuit 25, such as a CO₂ sensor 28 that measures CO₂ in gas expired by the infant 2, an O₂ sensor 27 that measures O₂ in gas inspired by the infant 2, a flow sensor 29 that measures gas flow within the breathing circuit 25, a pressure sensor 30 that measures pressure within the breathing circuit 25, or a temperature sensor 31 measuring temperature of expired and/or inspired gas within the breathing circuit 25. Each of the one or more aforementioned sensors may be contained in a respiration sensor device 26, such as a device positioned between the mask 36 and the breathing tube 38. For instance, the respiration sensor device 26 may be configured to communicate with one or more devices on the mobile newborn care bed 10, including the ventilator device 40 and/or the on-bed computing system 100. The respiration sensor device 26 may communicate wirelessly or by wires that extend to a receiving connector in the bassinet 12 (such as extending along and/or embedded into the breathing tube 38), or otherwise electrically connect to the ventilator device 40, the on-bed computing system 100, or another system that processes the sensor measurements. In other embodiments, the respiration sensor device 26 may communication the measurements to a wireless receiver associated with the ventilator device 40, the on-bed computing system 100, or another system that processes the sensor measurements.

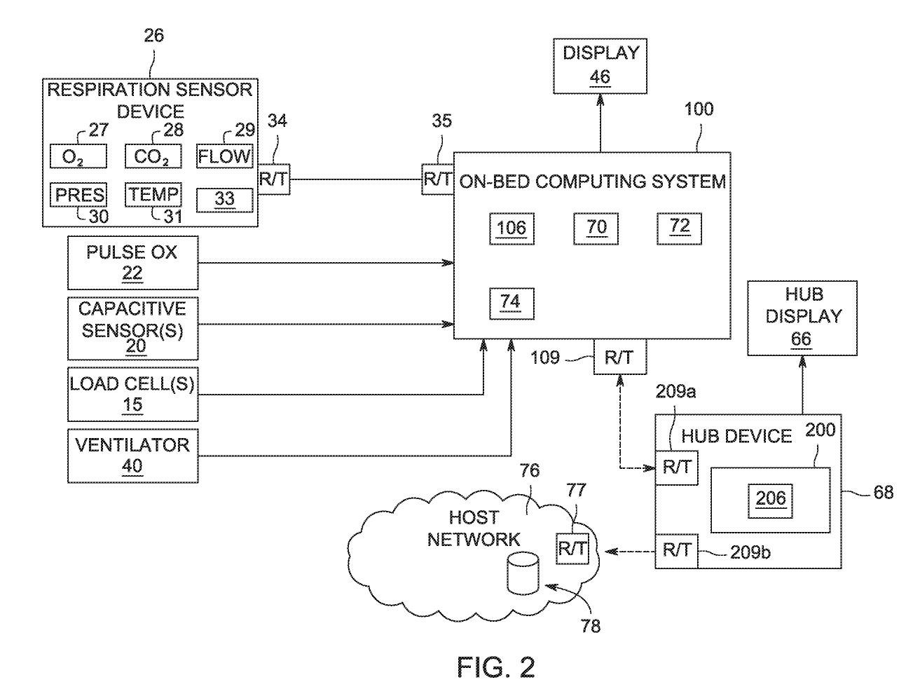

FIG. 2 schematically depicts an exemplary embodiment of the respiration sensor device 26 containing O₂ sensor 27 supplying O₂ measurement 90, CO₂ sensor 28 supplying CO₂ measurement 91, flow sensor 29 supplying flow measurement 92, pressure sensor 30 supplying pressure measurement 93, and temperature sensor 31 supplying temperature measurement 94. The mobile newborn care bed 10 may be configured with any one of the aforementioned sensors to provide respiration parameter measurements from the breathing circuit providing breathing gas to the infant 2, and such respiration parameter measurements may include, but are not limited to, the aforementioned measurements. The respiration sensor device 26 further includes a processor 33 that receives the measurements from each of the sensors 27-31 and communicates those measurements via wireless communication protocol to the on-bed computing system 100 through wireless receiver/transmitter 34. Transmissions from the wireless receiver/transmitter 34 are received by a wireless receiver/transmitter 35 associated with the on-bed computing system 100. The wireless receiver/transmitters 34 and 35 may communicate via any wireless protocol, and relatively short range protocols may be especially useful, such as Bluetooth, Bluetooth low energy (BLE), ANT, ZigBee, or a near field communication (NFC) protocol.

The respiration sensor device 26 may be configured to connect between the mask 36 and the breathing tube 38, and thus may have appropriate connecting means on either end to facilitate such connection within the breathing circuit 25. In another embodiment, the respiration sensor device 26 may be incorporated into the mask 36. In still other embodiments, each of the sensors 27-31 may be incorporated separately into the breathing circuit 25, such as into the mask 36, and each may communicate separately with the on-bed computing system 100 or other system configured to process the respiration related measurements.

Referring to FIGS. 2 and 3, the on-bed computing system 100 may include a software module stored in memory and executable on a processor 106 within the on-bed computing system 100—i.e., resuscitation module 72—configured to process one or more of the respiration parameter measurements 90-94 to generate respiratory information 96 regarding the respiratory status of the infant 2. For example, the resuscitation module 72 may determine respiratory information 96 including an inspired O₂ indicator, such as a fraction of inspired oxygen (FiO₂) value. Alternatively or additionally, respiratory information 96 determined by the resuscitation module 72 may include an end title CO₂ (etCO₂) based on the CO₂ measurements 91, title volume based on the flow measurements 92, and/or intake air pressure based on the pressure measurements 93. Alternatively or additionally, the resuscitation module 72 may utilize the temperature measurements 94 to determine the temperature of the inspired air and/or to determine information about the temperature of the infant 2. Any one of the aforementioned values may be included in the respiratory information 96 outputted by the resuscitation module 72, and such respiratory information 96 may be transmitted to the hub device 68 and/or the host network 76 for storage in the infant's medical record in database 78. Alternatively or additionally, some or all of the respiratory information 96 may be displayed on the digital display 46. In other embodiments, the resuscitation module 72 may be stored in memory and executable on a processor of the ventilator device 40, which may then communicate the respiratory information 96 to the on-bed computing system 100.

As shown in FIG. 1, the mobile newborn care bed 10 may include a battery 48 to power the various devices thereon, including some or all of the various sensing devices, the on-bed computing system 100, the ventilator device 40, and/or the digital display 46. The battery 48 may be positioned on the base frame portion 52a, for example, and in such a location to be easily accessed in order to recharge or replace the battery 48. The charge status of the battery 48 may be monitored by a power control module, such as may be provided separately from and in communication with, or otherwise incorporated into, the on-bed computing system 100. Further, the on-bed computing system 100 may provide a battery status notification, such as on digital display 46, regarding the charge of the battery 48 on the digital display 46 so that a clinician or other user will be able to determine the charge level of the battery 48.

The mobile newborn care bed 10 may be further configured to physically and/or wirelessly connect to a freestanding radiant warmer 60 to provide infant warming. For example, the radiant warmer 60 may be positioned away from the delivery location and the mobile newborn care bed 10 may be moved over to the radiant warmer 60 once the umbilical cord has been cut and the infant 2 can be moved away from the mother. The radiant warmer 60 may include a warming element 62, several of which are known and are utilized in the relevant art. The warming element 62 is suspended at an appropriate height and angle above the mobile newborn care bed 10 when the mobile newborn care bed 10 is moved to a predetermined location with respect to the radiant warmer 60, such as immediately adjacent to and/or connected to the front of the radiant warmer 60.

The radiant warmer 60 may include a base frame portion 64a and a vertical portion 64b. The vertical portion 64b may provide connection and support means for various electronic and/or electric elements, including the warming element 62 and/or a hub device 68 and hub display 66. The radiant warmer 60 may include reciprocal latching means 59 configured to connect to latching means 58 on the mobile newborn care bed 10. Accordingly, the mobile newborn care bed may be securely yet removably attached to the radiant warmer 60. For example, the reciprocal latching means 59 may be provided on a base frame portion 64a of the radiant warmer 60 at a location that conveniently connects to the base frame portion 52a of the mobile newborn care bed 10. The base frame portion 64a of the radiant warmer 60 may be equipped with wheels 65, and thus the radiant warmer 60 may be moved, and also the mobile newborn care bed 10 and radiant warmer 60 may be movable as a connected unit.

The radiant warmer 60 may be powered by a battery and/or by main electric power, such as through an AC wall outlet. Further, such power may be supplied to one or more devices on the mobile newborn care bed 10 when it is attached to the radiant warmer 60. For example, power transfer may occur from the radiant warmer 60 to the battery 48 in order to charge the battery 48. In just one embodiment, such power transfer connection may be provided through the latching means 58 and 59 connecting the respective devices.

Alternatively or additionally to the radiant warmer 60, the mattress 18 of the mobile newborn care bed 10 may be heated, such as by one or more heating elements 50 incorporated therein. The heating elements 50 may be any of various heating element-types known in the art, such as heating wires or coils commonly incorporated in electric heating blankets, flexible carbon-fiber heating tape, or heated water circulated through flexible coils. The heating elements 50 may be powered and controlled by a heating control unit 49, which may be incorporated into the mattress 18 or other location on the bassinet 12, with a power connection to the bassinet 12 to obtain power from the battery 48 or other power source. The heating control unit 49 may control the heating element(s) 50 based on input from one or more temperature sensors 57 placed on or adjacent to the heating element(s) 50, such as on or embedded in the top surface 18a of the mattress 18, to sense the temperature of the heating elements. For example, the heating control unit 49 may control the heating element(s) 50 heat the mattress 18 to approximately 98.6 degrees so that placing the infant 2 on the mattress 18 does not contribute to cold stress experienced by the baby. Alternatively, the mattress 18 may be heated to slightly above body temperature, such as to 90 degrees, to warm the infant 2. The heating control unit 49 may communicate with the on-bed computing system 100, and the digital display 46 may provide a user interface for the heating control unit 49, such as to control the temperature of the mattress 18 and/or to display error messages or alerts from the heating control unit 49 regarding the function of the heating system for the mattress 18.

As illustrated schematically in FIG. 2, the on-bed computing system 100 may connect wirelessly to hub device 68, which may in turn connect wirelessly to host network 76. The hub device 68 may be on the radiant warmer 60 as shown in FIG. 1 or may be positioned at any location within the communication distance of the mobile newborn care bed 10. Alternatively, the hub device 68 may be provided by a mobile computing device, such as a laptop, tablet, smart phone, or the like. For example, a software application may be provided to allow a clinician's tablet or smart phone to act as the hub device 68. In still other embodiments, the hub device 68 may be a fetal monitoring unit, and thus the mobile newborn care bed 10 may communicate the cardiac information, respiratory information, etc. to the fetal monitoring unit for transmission to the host network 76. In such an embodiment, the fetal monitoring unit may also provide the digital display 46 to display some or all of the cardiac information, respiratory information, etc.

The hub device 68 has a hub computing system 200 equipped with a processor 206. The exemplary hub computing system 200 in the hub device 68 is equipped to communicate with the on-bed computing system 100 and the host network 76 via receiver/transmitters 209a and 209b respectively. The host network 76 comprises a wireless receiver/transmitter 77 to receive and transmit information to one or both of the hub device 68 and the on-bed computing system 100.

The hub computing system 200 may further be configured to control the hub display 66. In one embodiment, the on-bed computing system 100 and/or the hub computing system 200 may be configured to determine when the mobile newborn care bed 10 is in a predetermined position with respect to the radiant warmer 60, such as attached to the radiant warmer 60 via the respective latching means 58 and 59. For example, one of the mobile newborn care bed 10 and/or the radiant warmer 60 may be equipped with a proximity sensor to determine the distance between the bed 10 and warmer 60. In one such embodiment, the mobile newborn care bed 10 and/or the radiant warmer 60 may be equipped with a proximity sensor device or other proximity determination capability to determine that the mobile newborn care bed 10 is within a predetermined distance of the radiant warmer 60, such as in a position such that the warming element 62 is over the infant 2. For example, such proximity sensors may be incorporated in or near the latching means 58 and 59 and/or in the base frame portion 52a of the mobile newborn care bed 10 or in the base frame portion 64a of the radiant warmer. In still other embodiments, the on-bed computing system 100 and/or the hub computing system 200 may include a distance determination module that estimates the position of the mobile newborn care bed 10 with respect to the radiant warmer 60 based on amplitude of wireless communication signals between the respective receiver/transmitters 109 and 209a.

The on-bed computing system 100 and/or the hub computing system 200 on the hub device 68 may then control the hub display 66 to display the various monitoring information—e.g., cardiac information 82 and respiratory information 96—being determined for the infant 2. Additionally, when the hub display 66 is being operated, the digital display 46 on the mobile newborn care bed 10 may be turned off to conserve battery 48 charge. The on-bed computing system 100 and/or the hub computing system 200 may be configured to automatically provide such display operation. Likewise, one or more of the computing systems 100, 200 may be configured to automatically switch on the digital display 46 of the mobile newborn care bed 10 when the mobile newborn care bed 10 is de-latched or otherwise moved away from the radiant warmer 60. Display commands from the on-bed computing system 100 may be routed to the hub display 66 through the hub computing system 200. Alternatively or additionally, the on-bed computing system 100 may be configured to communicate wirelessly directly to the hub display 66, which may have its own wireless receiver/transmitter.

Wireless communication between the on-bed computing system 100, the hub device 68, and the host network 76 may be by any wireless protocols known in the relevant art. In the depicted embodiment, the on-bed computing system 100 has receiver/transmitter 109 configured to communicate with receiver/transmitter 209a on the hub device 68. The various receiver/transmitters 34, 35, 109, 209a, 209b, 77 may include separate receiving and transmitting devices or may include an integrated device providing both functions, such as a transceiver. The on-bed computing system 100 and hub device 68, via respective receiver/transmitters 109 and 209a, may be configured as medical body area network (MBAN) devices. In other embodiments, the receiver/transmitters 109 and 209a may communicate via other relatively short range radio protocols, such as Bluetooth, Bluetooth Low Energy (BLE), ANT, ZigBee, or NFC. In other embodiments, the communication may be via network protocols appropriate for longer-range wireless transmissions, such as on the wireless medical telemetry service (WMTS) spectrum or on a Wi-Fi-compliant wireless local area network (WLAN). In still other embodiments, the receiver/transmitters 109 and 209a may be capable of switching between two or more wireless communication protocols, such as to optimize data communication based on the situation. For example, the receiver/transmitters 109 and 209a may utilize a very short range protocol, such as an NFC protocol, if the mobile newborn care bed 10 is attached to the radiant warmer 60, and may user a longer range communication protocol, such as Bluetooth or Wi-Fi, when the mobile newborn care bed 10 is not attached to or is more than a predetermined distance from the radiant warmer 60.

Similarly, the hub device 68 may communicate with the host network 76 via receiver/transmitter 209b on the hub device 68 and receiver/transmitter 77 associated within or incorporated in the host network 76, such as to transmit the cardiac information 82, bradycardia event notification 83, respiratory information 96, weight 85, or apnea event notification 86. In other embodiments, the hub device 68 may be eliminated and the on-bed computing system 100 may communicate directly with the host network 76. Such transmission may be via network protocol appropriate for longer-range wireless transmissions, such as on the WMTS spectrum or on a WLAN, as described above.

The host network 76 may be, for example, a local computer network having servers housed within a medical facility where the infant 2 is born, or it may be a cloud-based system housed by a cloud computing provider. The host network 76 may include a medical records database 78 housing the medical records for the infant 2, which may be updated to store the information transmitted by the on-bed computing system 100 and/or the hub device 68. The host network 76 may further include other patient care databases which may be accessed by or through either of the on-bed computing system 100 or the hub computing system 200, such as an ECG database.

FIG. 3 provides a system diagram of an on-bed computing system 100 having cardiac module 70 executable to determine cardiac information 82, resuscitation module 72 executable to determine respiratory information 96, and load module 74 executable to determine a weight 85 for the infant 2 and/or detect an apnea event and generate a notification 86. Furthermore, the cardiac module 70 may be executable to store the cardiac information 82 in storage system 104 of the on-bed computing system 100 so that such information may be accessed at a later time, such as to generate trend plots. Likewise, resuscitation module 72 may be executable to store respiratory information 96 and/or the measurement data from the sensors; and load module 74 may store the load measurements 84 and/or weight 85 and apnea event notifications 86 in storage system 104 of the on-bed computing system 100 so that such information may be accessed at a later time, such as to generate trend plots. For example, such information may be accessed by the various modules and/or by clinicians to determine whether the infant 2 is ready for discharge or whether certain physiological indicators indicate that continued care is needed, such as whether the infant 2 is experiencing continued apnea events and/or bradycardia events.

On-bed computing system 100 includes a processor 106, storage system 104, software 102, and communication interface 108. The processor 106 loads and executes software 102 from the storage system 104, including the cardiac module 70, resuscitation module 72, and load module 74, which are applications within the software 102. Each of the modules 70, 72, 74 include computer-readable instructions that, when executed by the on-bed computing system 100 (including the processor 106), direct the processor 106 to operate as described herein.

Although the computing system 100 as depicted in FIG. 3 includes one software 102 encapsulating one cardiac module 70, one resuscitation module 72, and one load module 74, it should be understood that one or more software elements having one or more modules may provide the same operation. Similarly, while description as provided herein refers to a computing system 100 and a processor 106, it is to be recognized that the methods and systems described herein be executed using one or more processors, which may be communicatively connected, and such implementations are considered to be within the scope of the description.

The processor 106 can comprise a microprocessor and other circuitry that retrieves and executes software 102 from storage system 104. Processor 106 can be implemented within a single processing device but can also be distributed across multiple processing devices or sub-systems that cooperate in executing program instructions. Examples of processor 106 include general purpose central processing units, application specific processors, and logic devices, as well as any other type of processing device, combinations of processing devices, or variations thereof.

The storage system 104 can comprise any storage media, or group of storage media, readable by processor 106 and capable of storing software 102. The storage system 104 may include volatile and non-volatile, removable and non-removable media implemented in any method or technology for storage of information, such as computer-readable instructions, data structures, program modules, or other data. Storage system 104 can be implemented as a single storage device but may also be implemented across multiple storage devices or sub-systems. Storage system 104 may further include additional elements, such a controller capable of communicating with the processor 106.

Examples of storage media include random access memory, read only memory, magnetic discs, optical discs, flash memory, virtual memory, and non-virtual memory, magnetic sets, magnetic tape, magnetic disc storage or other magnetic storage devices, or any other medium which can be used to storage the desired information and that may be accessed by an instruction execution system, as well as any combination or variation thereof, or any other type of storage medium. Likewise, the storage media may be housed locally with the processor 106, or may be distributed in one or more servers, which may be at multiple locations and networked, such as in cloud computing applications and systems. In some implementations, the storage media can be a non-transitory storage media. In some implementations, at least a portion of the storage media may be transitory.

The communication interface 108 is configured to provide communication between the processor 106 and various other systems and devices, including to receive measurement information from the various sensors and communicate commands and information to the hub device 68 and/or host network 76. For example, communication interface 108 may control or include receiver/transmitter 35 that communicates with receiver/transmitter 34 on the respiration sensor device 26. Likewise, communication interface 108 may control or include receiver/transmitter 109 that communicates with the receiver/transmitter 209a on the hub device 68. Likewise, communication interface 108 may receive information from wired connections, such as from the pulse oximeter 22, capacitive sensors 20, load cell 15, and/or ventilator device 40. Likewise, communication interface 108 may communicate with the controller for the digital display 46.

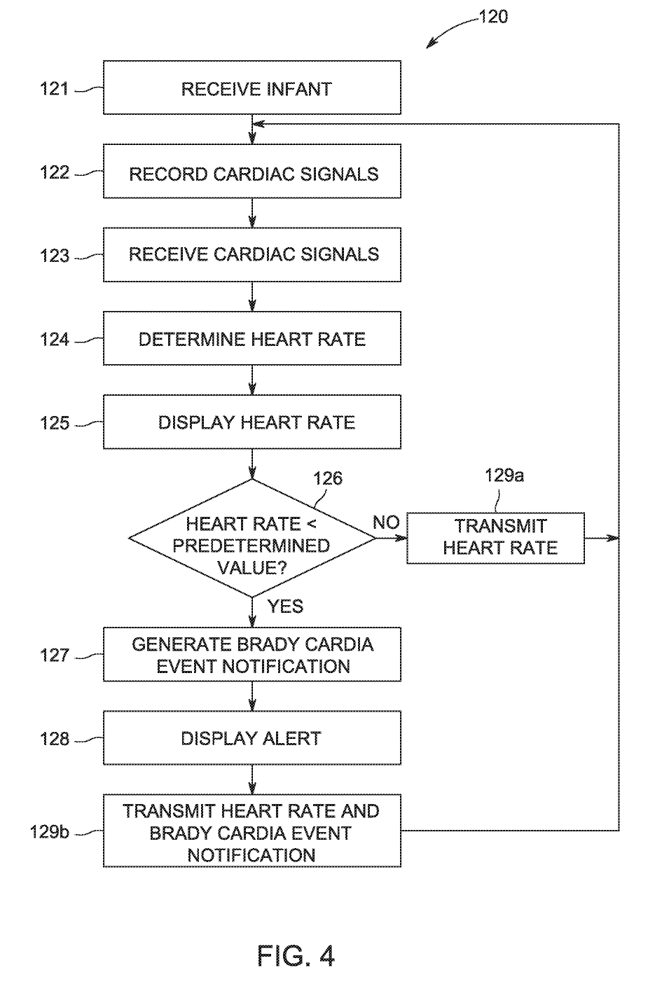

FIG. 4 depicts one embodiment of a method 120 of monitoring a newborn infant. The infant is received on the mobile newborn care bed 10 at step 121, such as placed on the mattress 18 having incorporated capacitive sensors 20. Cardiac potentials are recorded at step 122 via the capacitive sensors 20 to provide cardiac signals 81. The recorded cardiac signals 81 are received at step 123, such as at the on-bed computing system 100. The heart rate is determined at step 124, and the heart rate is displayed at step 125. Step 126 determines whether the heart rate is less than a predetermined value, such as a value set by a clinician that is appropriate based on the infant's age and size. If the heart rate is not less than the predetermined value then a bradycardia event is not detected and the heart rate is transmitted at step 129a such as to the hub device 68 or the host network 76. If the heart rate is less than the predetermined value then a bradycardia event is detected and a bradycardia event notification is generated at step 127. An alert may be displayed at step 128, such as on the digital display 46 on the mobile newborn care bed 10 and/or the hub display 66. The heart rate and bradycardia event notification are then transmitted at step 129b. In one embodiment, steps 123 through 129 are carried out by executing software of the cardiac module 70 on the processor 106 of the on-bed computing system 100. Furthermore, the cardiac module 70 may execute steps to determine other cardiac information 82 based on the cardiac signals 81, such as morphology-related values or trending assessments.

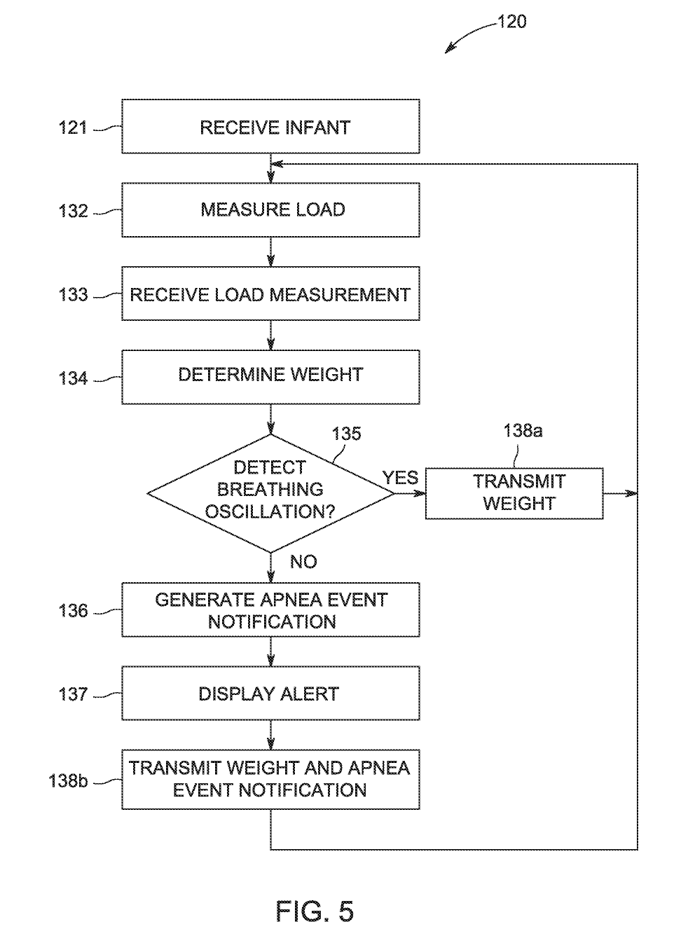

FIG. 5 depicts another embodiment of a method 120 of monitoring a newborn infant. When the infant is received on the mobile newborn care bed 10 at step 121, the load is measured at step 132 via one or more load cells 15. The load measurement is received at step 133, such as at the on-bed computing system 100. A weight 85 is determined at step 134 based on the load measurement 84. At step 135 it is determined whether breathing oscillations in the load measurement 84 are detected. Changes in distribution of load on the one or more load cells 15 will be caused by the up and down movements of the infant's diaphragm while the infant breaths. If such load distribution changes or oscillations in the load measurement 84 are detected, then it is determined that the infant 2 is breathing. For example, oscillations of a particular frequency, or threshold frequency, may be required at step 135. If the requisite oscillations are detected, then it is determined that an apnea event is not occurring and the weight 85 value can be transmitted at step 138a, such as to the hub device 68 or the host network 76. If the requirements of step 135 are not satisfied, then an apnea event is detected and an apnea event notification is generated at step 136. An alert may be displayed on the digital display 46 and/or the hub display 66 at step 137, and the weight and apnea event notification are transmitted at step 138b. In one embodiment, steps 133 through 138 may be carried out by executing instructions of the load module 74 on processor 106 of the on-bed computing system 100.

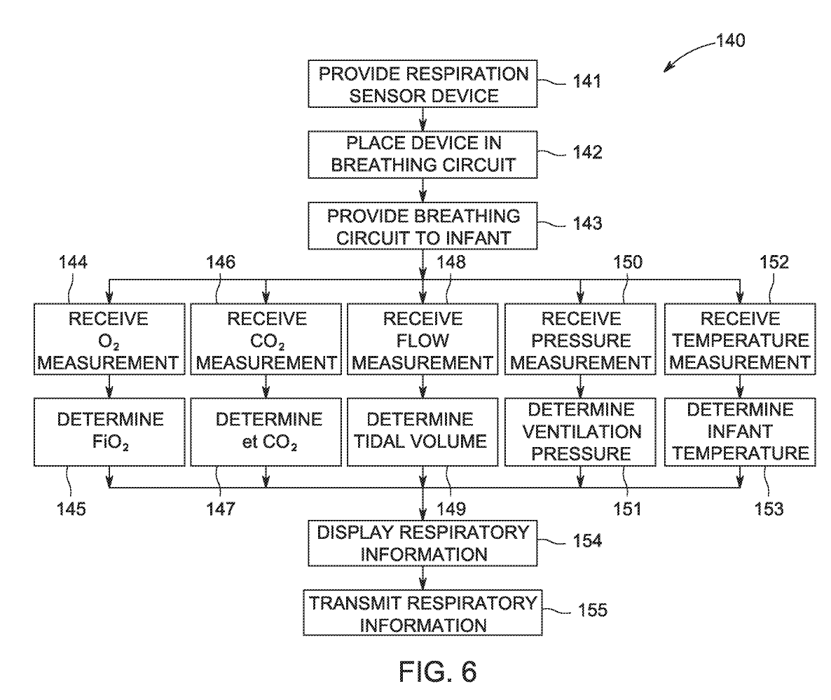

FIG. 6 depicts one embodiment of a method 140 of providing resuscitative care to an infant 2. A respiration sensor device 26 is provided at step 141, and the respiration sensor device 26 is placed in the breathing circuit 25 at step 142, such as between the mask 36 and breathing tube 38. The breathing circuit 25 is provided to the infant 2 at step 143, such as by placing the mask over the infant's nose and mouth. One or more respiration parameters are measured by various sensors within the breathing circuit 25, such as O₂, CO₂, flow rate, pressure, volume, and temperature. O₂ measurements are received at step 144, such as by a respiratory module 72 in the software of the on-bed computing system 100. The respiratory module 72 then determines an FiO₂ value at step 145 based on the O₂ measurements. Similarly CO₂ measurements are received at step 146 and an etCO₂ value is determined at step 147 based on the CO₂ measurements. Flow measurements are received at step 148 and a title volume is determined at step 149 based on the flow measurements. For example, a person having ordinary skill in the art will understand in light of this disclosure that the tidal volume may be calculated as an area under the flow curve formed by flow measurements recorded over a breath cycle of the infant 2. Pressure measurements are received at step 150, and a ventilation pressure is determined at step 151. Temperature measurements are received at step 152 and a temperature is determined at step 153 for the infant 2. Some or all of the forgoing respiratory information may be displayed at step 154, such as on the digital display 46 and/or the hub display 66. The respiratory information is transmitted at step 155, such as to the hub device 68 and/or the host network 76 as described herein. In one embodiment, steps 144 through 155 are carried out by executing instructions of the resuscitation module 72 on processor 106 of the on-bed computing system 100, or on a processor of the ventilator device 40. In another embodiment, one or more of the steps 144-153 are carried out within the respiration sensor device 26, such as by executing corresponding software instructions on the processor 33 thereof. The respective values generated at those steps may be transmitted to the on-bed computing system 100, which may then execute steps 154 and 155.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A mobile newborn care bed configured to be positioned at a delivery location of an infant, the mobile newborn care bed comprising:
a bassinette containing a mattress for receiving the infant;
at least two capacitive sensors incorporated in the mattress that record cardiac signals from the infant;
an on-bed computing system configured to receive the cardiac signals and determine a heart rate for the infant;
a battery that powers the on-bed computing system; and
a digital display that displays the heart rate, the digital display communicatively connected to the on-bed computing system.

2. The mobile newborn care bed of claim 1, wherein the at least two capacitive sensors are metal plates incorporated in the mattress connected to leadwires that transmit the cardiac signals to the on-bed computing system.

3. The mobile newborn care bed of claim 1, wherein the on-bed computing system includes a cardiac module executable on a processor for the on-bed computing system, the cardiac module executable to determine cardiac information based on the cardiac signals, wherein the cardiac information includes the heart rate.

4. The mobile newborn care bed of claim 3, wherein the on-bed computing system wirelessly transmits the cardiac information and/or the cardiac signals to at least one of a hub device and a host network.

5. The mobile newborn care bed of claim 3, wherein the cardiac module is executable to detect a bradycardia event based on the cardiac signals, generate a bradycardia event notification transmitted to the at least one of the hub device and the host network, and to generate a visual alert displayed on the digital display.

6. The mobile newborn care bed of claim 1, wherein the digital display is fixed to the bassinette and is powered by the battery.

7. The mobile newborn care bed of claim 1, wherein the on-bed computing system wirelessly transmits the heart rate and/or cardiac signals to a hub device on a radiant warmer, wherein the hub device is in data communication with and transmits the heart rate and/or cardiac signals to a host network.

8. The mobile newborn care bed of claim 7, wherein the on-bed computing system communicatively connects to a hub display on the radiant warmer when the mobile newborn care bed is in a predetermined position with respect to the radiant warmer.

9. The mobile newborn care bed of claim 1, further comprising:
a rigid base plate underneath and supporting the mattress;
at least one load cell under the rigid base plate that measures a load of the infant supported on the mattress; and
wherein the on-board computing system determines a weight based on the load measurement and controls the digital display to display the weight.

10. The mobile newborn care bed of claim 1, further comprising:
a respiration sensor device connectable to a breathing circuit for the infant to measure one or more respiration parameters and communicate the respiration parameter measurements to the on-bed computing system;
wherein the respiration sensor device includes one or more of an oxygen sensor, a carbon dioxide sensor, a flow sensor, and a pressure sensor, and the respiration parameter measurements correspondingly include one or more of an O₂ measurement, a CO₂ measurement, a flow measurement, and a pressure measurement; and
wherein the on-board computing system determines respiratory information based on the respiration parameter measurements and controls the digital display to display the respiratory information.

11. The mobile newborn care bed of claim 1, further comprising a pulse oximeter device configured to determine an SpO₂ value for the infant and transmit the SpO₂ value to the on-bed computing system, and wherein the on-board computing system controls the digital display to display the SpO₂ value.

12. A method of monitoring a newborn infant, the method comprising:
recording cardiac signals from the infant with two or more capacitive sensors incorporated in a mattress of a mobile newborn care bed;
receiving the cardiac signals at an on-bed computing system of the mobile newborn care bed;
determining a heart rate in the on-bed computing system based on the cardiac signals; and
displaying the heart rate on a digital display communicatively connected to the on-bed computing system.

13. The method of claim 12, further comprising wirelessly transmitting the cardiac signals and/or heart rate from the on-bed computing system to at least one of a host network and a hub device.

14. The method of claim 13 further comprising:
detecting a bradycardia event with the processor in the on-bed computing system based on the cardiac signals;
controlling the digital display with the on-bed computing system to display an alert; and
wirelessly transmitting a bradycardia event notification to the at least one of the host network and the hub device.

15. The method of claim 12, further comprising wirelessly transmitting the heart rate and/or cardiac signals from the on-bed computing system to the hub device on a radiant warmer, and wirelessly transmitting the heart rate and/or cardiac signals from the hub device to the host network.

16. The method of claim 15, further comprising:
determining at the on-bed computing system that the mobile newborn care bed is in a predetermined position with respect to a radiant warmer; and
transmitting a display command to a hub display on the radiant warmer from the on-bed computing system to display the heart rate and/or the cardiac signal when the mobile newborn care bed is in the predetermined position.

17. The method of claim 12, further comprising:
measuring a load of the infant with a load cell positioned under a rigid base plate supporting the mattress, the load cell communicatively connected to the on-bed computing system;
receiving the load measurement at the on-bed computing system;
determining a weight based on the load measurement; and
displaying the weight on the digital display.

18. The method of claim 17, further comprising:
detecting an apnea event based on the load information; and
wirelessly transmitting an apnea event notification to one of a hub device or a host network.

19. The method of claim 12, further comprising:
measuring one or more respiration parameters with a respiration sensor device communicatively connected to the on-bed computing system, wherein the respiration sensor device includes one or more of an oxygen sensor, a carbon dioxide sensor, a flow sensor, and a pressure sensor;
receiving the respiration parameter measurements at the on-bed computing system;
determining respiratory information with the on-bed computing system based on the respiration parameter measurements; and
displaying the at least a portion of the respiratory information on a digital display.

20. The method of claim 19, further comprising:
determining an SpO₂ value with a pulse oximeter communicatively connected to the on-bed computing system;
receiving the SpO₂ value at the on-bed computing system; and
displaying SpO₂ value on the digital display.

21. A mobile newborn care bed configured to be positioned at a delivery location of a newborn infant, the mobile newborn care bed comprising:
a bassinette containing a mattress that supports the infant;
a frame that supports the bassinette;
an on-bed computing system comprising a processor;
a flow sensor that measures a gas flow within a breathing circuit for the infant;
a CO₂ sensor that measures an expired CO₂ within the breathing circuit for the infant;
a resuscitation module executable on the processor of the on-bed computing system to receive the flow measurement and the CO₂ measurement and determine respiratory information for the infant; and
a digital display communicatively connected to the on-bed computing system that displays the respiratory information.

22. The mobile newborn care bed of claim 21, wherein the CO₂ sensor and the flow sensor are contained in a respiration sensor device communicatively connected to the on-bed computing system and connectable within a breathing circuit for the infant, wherein the respiration sensor device further comprises at least one of an O₂ sensor, a flow sensor, a pressure sensor, and a temperature sensor.

23. The mobile newborn care bed of claim 22, wherein the respiration sensor device connects between a breathing tube and a mask in the breathing circuit.

24. The mobile newborn care bed of claim 21, further comprising a respirator device and at least one gas supply tank supported on the frame and supplying gas to the breathing circuit for the infant.

25. A method of providing resuscitative care to a newborn infant, the method comprising:
measuring a gas flow with a flow sensor in a breathing circuit for the infant and communicating the flow measurement to an on-bed computing system;
measuring an expired CO₂ with a CO₂ sensor in the breathing circuit for the infant and communicating the CO₂ measurement to an on-bed computing system;
determine respiratory information for the infant with the on-bed computing system based on at least the flow measurement and the CO₂ measurement; and
displaying the respiratory information on a digital display communicatively connected to the on-bed computing system.

26. The method of claim 25, supplying a respiration sensor device communicatively connected to the on-bed computing system, the respiration sensor device containing at least the flow sensor and the CO₂ sensor and connectable within the breathing circuit for the infant.
